(19) United States
(12) Patent Application Publication
(10) Pub. No.: US 2004/0261324 A1
(43) Pub. Date: Dec. 30, 2004

(54) Self contained living environment

(76) Inventor: Eugene Tewari, Bethpage, NY (US)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/807,246

(22) Filed: Mar. 24, 2004

Related U.S. Application Data
(60) Provisional application No. 60/457,278, filed on Mar. 24, 2003.

(51) Int. Cl. E04B 1/34; E04G 11/04; E04H 15/20; C12N 15/09

(52) U.S. Cl. 52/2.18

(57) ABSTRACT

A self contained living environment formed from a frame and a tent material attached to the frame. The upright members of the frame are pre-inserted in loops on the outside of the tent material, thereby making it much easier to set up the living environment. An air purification system is coupled to the tent material and provides a continuous supply of filtered air to the interior of the living environment. The air purification system is either a positive pressure system or a negative pressure system. With this structure, the self contained living environment protects persons within the environment from exposure harmful biological and chemical agents by providing the interior of the living environment with filtered air through the positive pressure system, or protects persons outside the living environment from exposure harmful biological and chemical agents within the living environment by preventing contaminated air from escaping from the interior of the living environment through the negative pressure system.

### CROSS REFERENCE TO RELATED APPLICATION

The present invention claims priority to provisional Application Ser. No. 60/457,278, filed Mar. 24, 2003, and entitled “SELF CONTAINED LIVING ENVIRONMENT”, the entire disclosure of which is incorporated herein by reference.

### BACKGROUND OF THE INVENTION

1. Field of the Invention

The present invention relates to self contained living environments. In particular, the present invention relates to self contained living environments which are capable of filtering out harmful biological and chemical agents.

2. Description of the Related Art

There are many types of biological and chemical agents that can infect and cause severe harm to humans. For example, anthrax spores can infect humans through a cut or graze, in contaminated meat or by being inhaled. Anthrax is classified by the way it is contracted. Basically, there are three modes of contracting anthrax: cutaneous, gastro-intestinal and pulmonary or inhalation anthrax. Each of these modes has certain damages which result. For example, cutaneous contracted anthrax can cause damage to the skin tissues; gastro-intestinal contracted anthrax can cause fever, vomiting of blood and acute diarrhea; and pulmonary or inhalation contracted anthrax can cause hemorrhaging.

Currently, one solution to a biological or chemical attack is to utilize a gas mask or a chemical protection suit. The problem with these solutions, however, is that a gas mask alone might not protect a person from a biological or chemical agent that can enter the body through the skin, and the gas mask must be rated for the particular contaminant to be effective. Chemical protection suits can be complex and a hassle to wear. In addition, with chemical protections suits, a separate suit must be provided for each individual.

A self contained shelter has been proposed which is in the form of a tubular frame inside a tent. This construction, however, is not easily assembled. Because the tubular frame is assembled inside the tent, at least two people have to go inside the tent to assemble the tubular frame. This set up process takes up a lot of time in a situation when time is of the essence, i.e., in the case of a chemical or biological attack.

### SUMMARY OF THE INVENTION

It is therefore an object of the present invention to provide a self contained living environment which is capable of filtering out harmful biological and chemical agents.

It is also an object of the present invention to provide a self contained living environment which is easily and quickly assembled and eliminates the hassle and complexity of using a gas mask and/or a chemical protection suit.

It is a further object of the present invention to provide a self contained living environment which provides a safe haven for multiple persons in the event of a chemical or biological attack.

The above and related objects are accomplished by a novel self contained living environment which includes a specially engineered air purification system.

In a first embodiment of the present invention, the self contained living environment protects persons and family pets from coming into direct contact with toxic chemical agents and harmful pathogens. The self contained living environment of the first embodiment is preferably designed to afford a family of four to six a reasonable amount of living space, while allowing for the performance of basic human functions.

The first embodiment of the self contained living environment of the present invention is effective in protecting persons by maximizing air flow through the air purification system, providing the interior of the living environment with filtered air, and providing an easily assembled structure. The filtered air in the first embodiment is preferably supplied by a positive pressure system which operates to protect persons within the living environment from exposure to contaminated air outside the living environment. With the positive pressure system, air from outside the living environment is filtered and then supplied to interior of the living environment.

In a second embodiment of the present invention, the self contained living environment protects persons outside of the living environment from exposure to contaminated air within the living environment. The self contained living environment of the second embodiment is preferably designed to allow isolation of a contaminated person while allowing a reasonable amount of space for a physician to attend to care and examination of the contaminated person.

The second embodiment of the self contained living environment of the present invention is effective in protecting persons by maximizing air flow through the air purification system, protecting areas outside the living environment from contaminated air within the living environment, and providing an easily assembled structure. The filtered air in the second embodiment is preferably supplied by a negative pressure system. With the negative pressure system, air from inside the living environment is filtered and combined with fresh air from outside the living environment, then the combined filtered air is then supplied to interior of the living environment. This recycling and filtering of the air from within the living environment ensures that no contaminated air escapes from the living environment.

With either the positive pressure system or the negative pressure system, the air purification system of the present invention works by trapping and sterilizing biological and chemical agents. Preferably, the air purification system of the present invention utilizes a three-stage filter which includes a HEPA Filter portion which is at least 99.97% efficient in trapping particles down to at least 0.3 microns in size, a specially blended carbon filtration media portion for chemical and gaseous agents, and a UV-C germicidal filter portion.

This three stage filter provides a powerful combination of Carbon and HEPA filtration with UV sterilization to protect persons from three types of airborne threats, i.e., poisonous gases and chemicals, toxic spores such as anthrax, mold spores, and micro-organisms such as airborne bacteria and viruses.

Preferably, the self contained living environment of the present invention is formed from a frame and a tent material attached to the frame. The upright members of the frame preferably come pre-inserted in loops on the outside of the tent material, thereby making it much easier to set up the living environment. With this structure, the living environment can be set up within six to ten minutes. In addition, the entrance is preferably “I” shaped with a two-sided zipper for easy ingress and egress. Other entry styles, such as a roll-up flap can also be used.

The material of the tent is preferably a clear plastic material, such as PVC, which has an oxygen transmission rate and a water vapor transmission rate effective to prevent chemical and biological agents from entering into or escaping from the living environment through the material. Other materials, such as a spun-bonded polyolefin, with appropriate oxygen and water vapor transmission rates may also be used.

The frame and tent material of the self contained living environment of the present invention are preferably modular by design so that the living environment area can be easily expanded, if necessary.

With the above-described structure, the self contained living environment of the present invention is effective in protecting persons within the environment from exposure harmful biological and chemical agents by providing the interior of the living environment with filtered air through a positive pressure system, or is effective in protecting persons outside the living environment from exposure harmful biological and chemical agents within the living environment by preventing contaminated air from escaping from the interior of the living environment through a negative pressure system. With either the positive or negative pressure system, the system is easily assembled.

### BRIEF DESCRIPTION OF THE DRAWINGS

For the purpose of illustrating the invention, there is shown in the drawings a form which is presently preferred, it being understood, however, that the invention is not limited to the precise arrangement shown, wherein:

FIG. 1 shows an assembled self contained living environment according to a first embodiment of the present invention;

FIG. 2 shows the air purification system for use with the present invention; and

FIG. 3 shows an assembled self contained living environment according to a second embodiment of the present invention.

### DESCRIPTION OF THE PREFERRED EMBODIMENTS

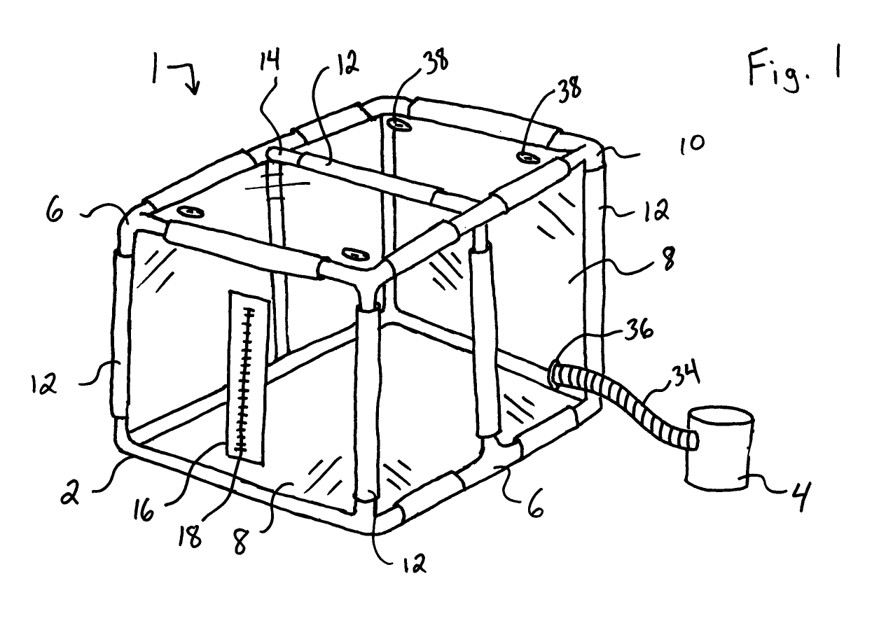

Referring now to the drawings, FIG. 1 shows a first embodiment of the self contained living environment 1 of the present invention.

The first embodiment of the self contained living environment 1 includes a living environment structure 2 and an air purification system 4 coupled to the structure 2. As shown in FIG. 1, the living environment of the present invention is formed from a frame 6 and a tent material 8 attached to the frame 6. Preferably, the frame 6 is made of 2 inch diameter PVC tube. Other tube materials and diameters, however, can be used. For example, if a larger size living environment is desired, the frame 6 is preferably made from aluminum tubing. This provides a strong, light weight structure.

The upright members 10 of the frame 6 preferably come pre-inserted in loops 12 on the outside of the tent material 8. Thus, the frame 6 is provided on the outside of the living environment. Because the upright members 10 are pre-inserted and the frame 6 is on the outside of the living environment, the structure can be easily set up within minutes. It is also preferred, as shown in FIG. 1, that the top cross-members 14 be pre-inserted into loops 12 on the outside of the living environment so that the roof portion of the tent material 8 is supported.

The frame 6 and tent material 8 which forms the self contained living environment 1 is preferably modular in design. This allows for the expansion of the space within the structure to accommodate additional persons.

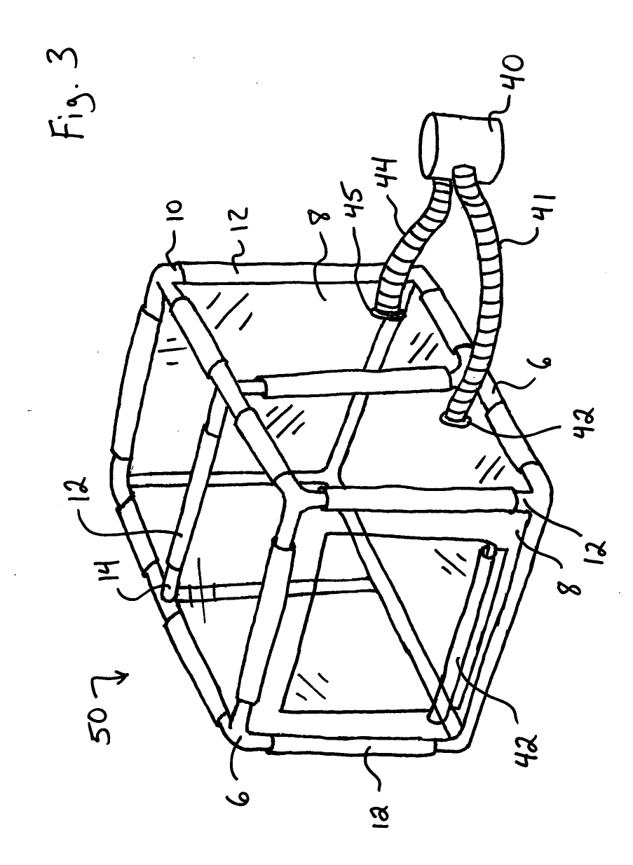

In addition, as shown in FIG. 1, the entrance 16 to the living environment is preferably “I” shaped with a two-sided zipper 18 for easy ingress and egress. Other entrance shapes, such as triangle, semi-circular, etc., can also be used. In addition, other entry styles, such as a roll-up flap (see FIG. 3) can also be used.

The tent material 8 is preferably a clear plastic material, such as PVC, which has an oxygen transmission rate and a water vapor transmission rate effective to prevent the chemical and biological agents from entering the living environment through the material.

As shown in FIG. 1, the air purification system 4 used in the first embodiment is a positive pressure system. The positive pressure system maximizes air flow through the air purification system 4 and into the interior of the living environment, thereby operating to protect persons within the living environment from exposure to contaminated air outside the living environment.

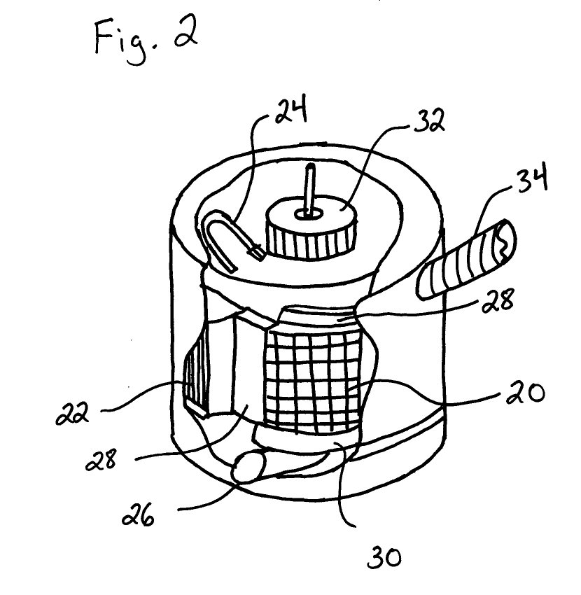

With the positive pressure system, air from outside the living environment is filtered and then supplied to interior of the living environment. As shown in FIG. 2, the air purification system 4 of the present invention preferably utilizes a three stage filter that works by trapping and sterilizing biological and chemical agents. Preferably, the three stage filter of the present invention includes a specially blended carbon filtration media portion 20 for chemical and gaseous agents, a HEPA Filter portion 22 which is at least 99.97% efficient in trapping particles down to at least 0.3 microns in size, and a UV-C germicidal filter portion 24. Each of these filter portions will be described in further detail hereinbelow.

As shown in FIGS. 1 and 2, in the positive pressure system air from outside the living environment enters the air purification system 4 through an opening 26 in the air purification system. The opening 26 is preferably provided in the bottom of the air purification system 4, but can also be configured as a duct which routs air from a desired location into the air purification system. After the air enters the air purification system 4, gases, chemicals, odors and fumes are filtered through the carbon filtration media portion 20. The carbon filtration media portion 20 of the air purification system 4 preferably uses about 15 to 18 pounds of carbon for filtering chemical and gaseous agents. Preferably, the carbon filter media is nuclear grade activated carbon. The active carbon filter works by absorbing the contaminated air, which passes through the filter, depositing pollutant molecules within the activated carbon. In other words, the carbon acts like a sponge, absorbing chemicals, gases and odors.

After exiting the carbon filtration media portion 20, the air is then routed through the HEPA filter portion 22 for particle removal. In the HEPA filter portion 22, particles such as dust, dust mites, pollen, antigens & other airborne particles are filtered. The HEPA filter portion 22 is preferably rated as being at least 99.97% efficient in trapping particles down to at least 0.3 microns in size. The HEPA filter used in the air purification system of the present invention is also preferably effective at removing solid as well as liquid particles.

After exiting the HEPA filter 22, the air is then routed through a UV-C germicidal filter portion 24. The UV-C germicidal filter portion 24 preferably comprises a UV lamp which kills pathogens after a predetermined amount of exposure time. The UV-C filter 24 is used to deactivate the DNA of bacteria, viruses and other pathogens, and thus destroy their ability to multiply and cause disease. Specifically, UV-C light emitted by the UV-C filter portion 24 causes damage to the nucleic acid of micro-organisms by forming covalent bonds between certain adjacent bases in the DNA of the bacteria, viruses and other pathogens. The formation of such bonds prevents the DNA from being “unzipped” for replication, and the organism is unable to reproduce. In most instances, when the organism tries to replicate after the formation of such covalent bonds, it dies.

Pathogens such as TB, smallpox and anthrax require less than a 9,000 microwatt germicidal dose to effectively inactivate or kill them. Preferably, the UV-C filter portion 24 used in the air purification system 4 is a four lamp system. The four lamp system is preferably rated to supply over 150,000 microwatts/cm² at a fan speed of 100 cfin. With such a system, as fan speed decreases, the UV dosage increases in proportion to the airflow change. For example, if the fan speed were reduced to 50 cfin, the UV dosage would be over 300,000 microwatts/cm².

In addition to the above filters, the air purification system 4 is also preferably provided with two anti-microbial filters 28 and a pre-filter 30 as shown in FIG. 2. The two anti-microbial filters 28 are placed inside and outside the carbon filter 20 so as to prevent small biological entities from growing on or in the canister for the carbon filter. The pre-filter 30 is formed from a special polyester fabric that traps large particles. The pre-filter 30 assists in keeping the HEPA filter 22 from being saturated by large particles, thereby making the HEPA filter 22 much more efficient.

Further, the air purification system 4 is preferably provided with a 300 CFM fan 32 which establishes a positive airflow within the living environment. After the positive airflow is established, the fan speed is preferably lowered to about 50 cfin, thereby maintaining the positive airflow and, in combination with the vents in the tent material, prevents contaminants from entering the living environment. As shown in FIG. 2, the fan 32 operates to deliver the “cleaned” air to the living environment regardless of whether the air purification system is located above or below ground. The “cleaned” air is then expelled from the air purification system via a duct 34 and routed into the living environment through an opening 36 in the tent material 8.

The air purification system 4 is also preferably designed so as to have a laboratory seal which assures that the “cleaned” air routed into the living environment is properly and effectively filtered. The laboratory seal guarantees that the contaminated air will not bypass the filters.

With the positive pressure system, the tent material 8 preferably has at least one opening 38 which operates as a one-way vent and cooperates with the air purification system 4 for air release so that a positive pressure can be maintained within the living environment. Preferably, the tent material 8 is provided with four one-inch openings 38 at the top four comers of the structure.

FIG. 3 shows a second embodiment of the self contained living environment 50 of the present invention. The second embodiment of the self contained living environment includes a living environment structure similar to that described above in relation to the embodiment of FIG. 1 (i.e., a frame 6 inserted into loops 12 on the outside of the tent material 8). Accordingly, where the structure does not differ from the first embodiment, like reference numerals represent like components, and the description thereof is not repeated.

The main differences between the structure shown in FIG. 1 and that of FIG. 3 is that the second embodiment uses a negative pressure air purification system 40, and the tent material 8, due to use of the negative pressure system, does not include any one-way vents. In addition, the entrance to the living environment shown in FIG. 3 is a roll-up flap 42. Other entry types, however, can be used.

As with the first embodiment, the frame 6 and tent material 8 which forms the self contained living environment 50 of the second embodiment is preferably modular in design to allow for the expansion of the space within the structure.

The second embodiment of the self contained living environment 50 of the present invention is designed to protect persons outside of the living environment from exposure to contaminated air within the living environment. The self contained living environment of the second embodiment is preferably designed to allow isolation of a contaminated person while allowing a reasonable amount of space for a physician to attend to care and examination of the contaminated person.

The air purification system 40 shown in FIG. 3 is a negative pressure system and utilizes a similar three stage filter to that shown in FIG. 2. Accordingly, the three stage filter in the negative pressure system also works by trapping and sterilizing biological and chemical agents through the use of a specially blended carbon filtration media portion for chemical and gaseous agents, a HEPA Filter portion which is at least 99.97% efficient in trapping particles down to at least 0.3 microns in size, and a UV-C germicidal filter portion. As these components were described above with respect to FIG. 2, the description thereof is not repeated.

With the negative pressure system, air from inside the living environment is routed to the air purification system 40 through a first duct 41 coupled to a first port 42 in the tent material 8. In addition, air from outside the living environment is routed to the air purification system 40 by an opening in the air purification system similar to that described above with respect to FIG. 2. The air from inside the living environment and the air from outside the living environment are combined and then “cleaned” as described above with reference to FIG. 2. Thereafter, the combined “cleaned” air is then routed to the living environment through a second duct 44 coupled to a second port 45 in the tent material. This recycling and filtering of the air from within the living environment ensures that no contaminated air escapes from the living environment.

With the above-described structures, the self contained living environment of the present invention is effective in protecting persons within the environment from exposure harmful biological and chemical agents by providing the interior of the living environment with filtered air through a positive pressure system, or is effective in protecting persons outside the living environment from exposure harmful biological and chemical agents within the living environment by preventing contaminated air from escaping from the interior of the living environment through a negative pressure system. In addition, with either the positive or negative pressure system, the self contained living environment structure is easily assembled.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A self contained living environment comprising:
a living environment structure, the living environment structure including a tent material defining an interior space and a frame provided on the exterior of the tent material to support the tent material; and
an air purification system coupled to the living environment structure so as to provide filtered air into the interior space.

2. The self contained living environment according to claim 1, wherein an exterior surface of the tent material includes loops, the loops being coupled to the frame.

3. The self contained living environment according to claim 2, wherein the frame includes upright members and at the upright members are pre-inserted into the loops.

4. The self contained living environment according to claim 2, wherein the frame includes top cross-members and at the top cross-members are pre-inserted into the loops.

5. The self contained living environment according to claim 1, wherein the frame and the tent material are modular.

6. The self contained living environment according to claim 1, wherein the tent material includes an opening for ingress to and egress from the interior space.

7. The self contained living environment according to claim 6, wherein the opening opened and closed with a zipper.

8. The self contained living environment according to claim 6, wherein the opening is a roll-up flap.

9. The self contained living environment according to claim 1, wherein the tent material has an oxygen transmission rate and a water vapor transmission rate effective to prevent chemical and biological agents from entering the interior space through the tent material.

10. The self contained living environment according to claim 9, wherein the tent material is preferably a clear plastic material.

11. The self contained living environment according to claim 1, wherein the air purification system is a positive pressure system which filters air from outside the interior space and supplies the filtered air to the interior space.

12. The self contained living environment according to claim 1, wherein the air purification system is a negative pressure system which filters both air from inside the interior space and air from outside the interior space, and supplies the filtered air to the interior space.

13. The self contained living environment according to claim 1, wherein the air purification system includes a three stage filter that works by trapping and sterilizing biological and chemical agents

14. The self contained living environment according to claim 13, wherein the three stage filter includes a carbon filtration media portion, a HEPA Filter portion, and a UV-C germicidal filter portion.

15. The self contained living environment according to claim 14, wherein gases, chemicals, odors and fumes are filtered through the carbon filtration media portion.

16. The self contained living environment according to claim 15, wherein the carbon filtration media portion is nuclear grade activated carbon.

17. The self contained living environment according to claim 14, wherein the HEPA Filter portion is at least 99.97% efficient in trapping particles down to at least 0.3 microns in size.

18. The self contained living environment according to claim 17, wherein the HEPA Filter portion is effective at removing solid as well as liquid particles.

19. The self contained living environment according to claim 14, wherein the UV-C germicidal filter portion comprises a UV lamp which kills pathogens after a predetermined amount of exposure time.

20. The self contained living environment according to claim 19, wherein the UV-C filter portion is a four lamp system rated to supply over 150,000 microwatts/cm² at a fan speed of 100 cfin.

21. The self contained living environment according to claim 13, wherein the air purification system further includes at least one anti-microbial filter and a pre-filter.

22. The self contained living environment according to claim 1, wherein the tent material has at least one opening which operates as a one-way vent and cooperates with the air purification system for air release so that a positive pressure can be maintained within the interior space.

23. The self contained living environment according to claim 22, wherein the tent material is provided with four one-inch openings at the top four corners of the structure.

24. A self contained living environment comprising:
a living environment structure comprising:
a tent material having an exterior surface with a plurality of loops attached thereto, the tent material defining an interior space; and
a plurality of frame members, each frame member coupled to the tent material by a respective loop of the plurality of loops so as to support the tent material exterior to the interior space; and
an air purification system coupled to the living environment structure so as to provide filtered air into the interior space,
wherein the tent material has an oxygen transmission rate and a water vapor transmission rate effective to prevent chemical and biological agents from entering the interior space through the tent material.

25. The self contained living environment according to claim 24, wherein the tent material includes an opening for ingress to and egress from the interior space.

26. The self contained living environment according to claim 24, wherein the air purification system is a positive pressure system which filters air from outside the interior space and supplies the filtered air to the interior space.

27. The self contained living environment according to claim 26, wherein the tent material has at least one opening which operates as a one-way vent and cooperates with the air purification system for air release so that a positive pressure can be maintained within the interior space.

28. The self contained living environment according to claim 24, wherein the air purification system is a negative pressure system which filters both air from inside the interior space and air from outside the interior space, and supplies the filtered air to the interior space.

29. The self contained living environment according to claim 24, wherein the air purification system includes a three stage filter that works by trapping and sterilizing biological and chemical agents

30. The self contained living environment according to claim 29, wherein the three stage filter includes a carbon filtration media portion, a HEPA Filter portion, and a UV-C germicidal filter portion.
